(19) United States
(12) Patent Application Publication
(10) Pub. No.: US 2012/0046653 A1
(43) Pub. Date: Feb. 23, 2012

(54) PULSED THERAPEUTIC LIGHT SYSTEM AND METHOD

(75) Inventors: Richard Shaun Welches, Manchester, NH (US); Mirko Mirkov, Chelmsford, MA (US); Rafael Armando Sierra, Palmer, MA (US)

(73) Assignee: CYNOSURE, INC., Westford, MA (US)

(21) Appl. No.: 13/254,477

(22) PCT Filed: Mar. 4, 2010
(86) PCT No.: PCT/US10/26211

§ 371 Date: Oct. 6, 2011

Related U.S. Application Data
(60) Provisional application No. 61/157,862, filed on Mar. 5, 2009.

(51) Int. Cl. A61N 5/06; A61B 18/18

(52) U.S. Cl. 606/13; 607/90

(57) ABSTRACT

In one aspect, a method is disclosed of delivering therapeutic light to a treatment region to effect heating in the treatment region. The method includes providing a light emitting diode and driving the light emitting diode to generate therapeutic light during a pulse period. The method further includes directing the therapeutic light to the treatment region at an average power density of about 200 W/cm² or more at the treatment region during the pulse period and maintaining the light emitting diode in a non-emitting state for a recovery period which is longer than the pulse period.

### CROSS-REFERENCE TO RELATED PATENT APPLICATIONS

The present application claims benefit of U.S. Provisional Patent Application Ser. No. 61/157,862 filed Mar. 5, 2009, the contents of which is incorporated by a reference herein in its entirety.

### BACKGROUND

Many light based dermatological treatments are performed currently with lasers or pulsed flashlamps/Intense Pulsed Light (IPL). Some professional grade devices used in dermatological treatments require peak power density between, e.g., 200 and 20,000 W/cm² delivered in pulse durations between, e.g., 0.5 and 50 ms.

Light emitting diodes (LED) are semiconductor light sources that are typically lower cost and more robust than lasers and IPLs. Their drawback is their relatively lower power density. The peak power specifications of some available state of the art LED devices (e.g. 130 W/cm², reported in Journal of Display Technology 2007, Vol. 3, pages 160-175) approach the low end of the peak power density range for lasers or IPLs. Lower power LED devices are currently used for non-thermal dermatological treatments like photobiomodulation (McDaniel, U.S. Pat. No. 6,663,659). Photobiomodulation and similar low level light therapies are non-thermal modalities based on photo-biological processes that can be induced at normal tissue temperatures. Typical state of the art LEDs do not have enough power density to achieve effective thermal treatment modalities where at least a subset volume of the skin is at an elevated temperature above normal tissue temperature. Thus, there is currently a need in the art for high power LEDs suitable for dermatological treatments.

### SUMMARY

In one aspect, a method is disclosed of delivering therapeutic light to a treatment region to effect heating in the treatment region. The method includes providing a light emitting diode and driving the light emitting diode to generate therapeutic light during a pulse period. The method further includes directing the therapeutic light to the treatment region at an average power density of about 200% W/cm² or more at the treatment region during the pulse period and maintaining the light emitting diode in a non-emitting state for a recovery period which is longer than the pulse period.

In some embodiments, the pulse period is about 50 ms, 10 ms, 1 ms, or less. In some embodiments, the duration of the recovery period is, at least 10 times, 50 times, or 100 times the duration of the pulse period.

In one embodiment, the method further includes, during the pulse period, repetitively cycling the light emitting diode between a light emitting state and a non emitting state to produce a series of sub-pulses of therapeutic light. In another embodiment, the step of repetitively cycling includes repetitively cycling the light emitting diode with a duty cycle of about 50% or less. In yet another embodiment, the sub-pulses have a duration of about 1 millisecond or less.

In some embodiments, the light emitting diode is characterized by a catastrophic, failure power level, and wherein, during the subpulses, the light emitting diode is operated at a power level equal to about 30%, 70%, 90%, or more of the catastrophic failure power level.

In some embodiments, the step of directing the therapeutic light to the treatment region at an average power density of about 200 W/cm̂2 or more at the treatment region during the pulse period comprises directing the therapeutic light to the treatment region at an average power density of about 500 W/cm̂2, 1,000 W/cm̂2, 5,000 W/cm̂2, 10,000 W/cm̂2, 20,000 W/cm̂2 or more at the treatment region during the pulse period.

In one embodiment, the method further includes dissipating heat generated by the light emitting diode using at least one heat sink in thermal communication with the light emitting diode. In another embodiment, the light includes infrared light.

In one embodiment, the method further includes effecting heating of the treatment region using the therapeutic light, and wherein the treatment region includes tissue, and wherein the heating results in a chemical or physical change to the tissue. In another embodiment, the chemical or physical change includes at least one selected from the list consisting of ablation, cauterization, inciting, shrinkage, lipolysis, and thermal damage, and wherein the tissue includes collagen, and the chemical or physical change includes heat induced collagen shrinkage.

In some embodiments, the treatment region is an external or an internal region of a human or animal subject. In another embodiment, the target region has a characteristic size of 1 cm or greater.

In another aspect, an apparatus is disclosed for delivering therapeutic light to a treatment region to effect heating in the treatment region. The apparatus includes a light emitting diode, a controller configured to drive the light emitting diode to generate therapeutic light during a pulse period and maintaining the light emitting diode in non-emitting state for a recovery period which is longer than the pulse period, and an optical delivery system configured to direct the therapeutic light to the treatment region at an average power density of about 200 W/cm² or more at the treatment region during the pulse period.

In some embodiments, the pulse period is about 50 ms, 10 ms, 1 ms, or less. In some other embodiments, the duration of the recovery period is at least 10, 50, or 100 times the duration of the pulse period.

In one embodiment, the controller is configured to drive the light emitting diode to, during the pulse period, repetitively cycle the light emitting diode between a light emitting state and a non-emitting state to produce a series of sub-pulses of therapeutic light. In another embodiment, the controller is configured to drive the light emitting diode to repetitively cycle the light emitting diode between the emitting and non-emitting states with a duty cycle of about 50% or less during the pulse period.

In one embodiment, the subpulses have a duration of about 1 millisecond or less.

In some embodiments, the light emitting diode is characterized by a catastrophic failure power level, and wherein, during the subpulses, the light emitting diode is operated at a power level equal to about 30%, 70%, 90% or more of the catastrophic failure power level.

In some embodiments, the optical delivery system directs the therapeutic light to the treatment region at an average power density of about 500 W/cm̂2, 1,000 W/cm̂2, 5,000 W/cm̂2, 10,000 W/cm̂2, 20,000 W/cm̂2 or more at the treatment region during the pulse period.

In one embodiment, the apparatus further includes at least one heat sink in thermal communication with the light emitting diode for dissipating heat generated by the light emitting diode. In another embodiment, the therapeutic light includes infrared light.

In one embodiment, the optical delivery system includes a fiber optic configured to receive therapeutic light from the light emitting diode at a proximal end and transmit the light to a distill end to illuminate the treatment region.

In one embodiment, the apparatus includes a surgical handpiece which is configured for insertion into a human or animal subject to deliver therapeutic light to an internal region.

In another embodiment, the apparatus includes a handpiece for delivering therapeutic light to external region of a human or animal subject. In yet mother embodiment, the target region has a characteristic size of 1 cm or greater.

In another aspect, an apparatus is disclosed for delivering therapeutic light to a treatment region to effect heating in the treatment region. The apparatus includes a plurality of light emitting diodes, a controller configured to drive each respective light emitting diode to generate therapeutic light during a respective pulse period and maintaining each respective light emitting diode in a non-emitting state for a respective recovery period which is longer than the pulse period, and at least one optical delivery system configured to direct therapeutic light from the light emitting (nodes to the treatment region at an average power density of about 200 W/cm² at the treatment region during a treatment period.

In one embodiment, the respective pulse periods of the respective light emitting diodes are staggered relative to each other during the treatment period. In another embodiment, at least one of the plurality of diodes in its respective pulse period during substantially the entire treatment period.

In one embodiment, the duration of the respective pulse period of each light emitting diode is 50 ms or less.

In another embodiment, the duration of the respective pulse period of each light emitting diode is about 10 times the duration of the recovery period or more.

### BRIEF DESCRIPTION OF THE DRAWINGS

FIGS. 1-3 show therapeutic light delivery systems.

FIG. 4 is a schematic of a pulsed LED therapeutic light source.

FIG. 5 illustrates a pulse scheme for a pulsed LED therapeutic light source.

FIG. 6 illustrates an array of pulsed LED therapeutic light sources

### DETAILED DESCRIPTION

Referring generally to the figures, systems and methods for delivering therapeutic light to a treatment region using light emitting diodes (LEDs) is shown. The LEDs may be driven in a pulsed mode to provide power that is above the normal continuous operating level for the LEDs. The LEDs may be turned on at the increased operating level for a short period of time (e.g., from 0.5 to 50 ms), followed by a longer period of time (e.g., hundreds of milliseconds) between pulses, allowing the accumulated heat from the on time to dissipate. This allows for the LED source to be used a suitable light source for a light based dermatological treatments in place of lasers and IPLs. The therapeutic light may heat the treatment region such that the heating results in a chemical or physical change to the tissue in the treatment region (e.g., an ablation, cauterization, melting, shrinkage, lipolysis, thermal damage, or another chemical or physical change). The tissue may include collagen, and the chemical or physical changes may include collagen shrinkage. The heating may include raising the tissue above its normal temperature e.g. by 5 degrees C. or more, by 10 degrees C. or more, etc.

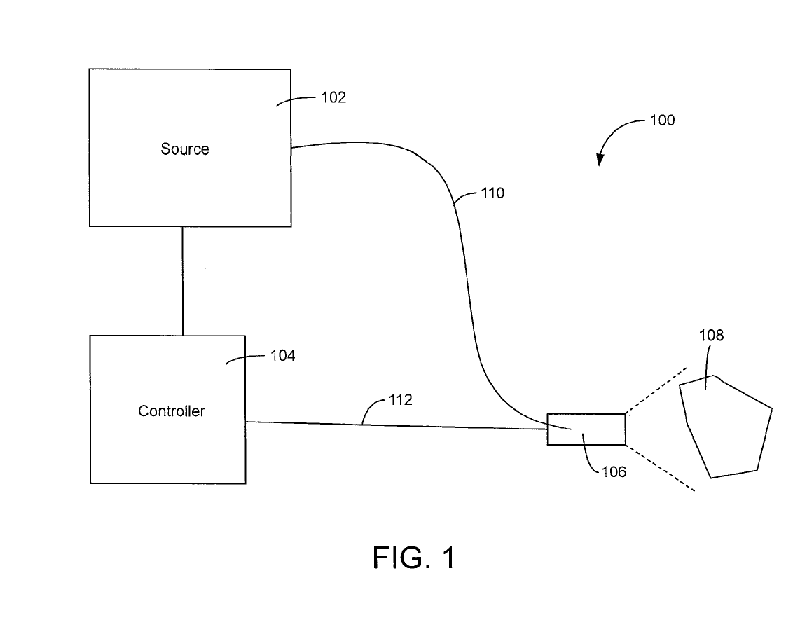

Referring to FIG. 1, an apparatus 100 for delivering therapeutic light to a treatment region is shown, according to an exemplary embodiment. Apparatus 100 includes source 102, controller 104, and handpiece 106. Source 102 may be a power source for providing power to an LED of handpiece 106 and may be connected to handpiece 106 via optical fiber 110.

Handpiece 106 may include one or more LEDs for providing a light source for providing therapeutic light (e.g., infrared light) for a treatment region 108 (e.g., an external region or internal region of a human or animal subject) for heating treatment region 108. Handpiece 106 may be operated by a user of apparatus 100 for providing treatment region 108 with therapeutic light from handpiece 106. According to various exemplary embodiments, handpiece 106 may be of any shape, size, or configuration for providing therapeutic; light from the LEDs. Handpiece 106 may be configured to provide therapeutic light for a target region of a specific size (e.g., 1 cm or greater, according to an exemplary embodiment). According to an exemplary embodiment, handpiece 106 may be similar to the handpiece disclosed in PCT Publications WO 2008/007218 and WO 2008/153999, the entire contents of each of which is incorporated by reference herein in its entirety.

Controller 104 may be configured to control the amount of light emitted by the LED and the time intervals for which light is emitted b′ the LED. For example, controller may control the voltage or current applied to drive the LED to emit light. Controller 104 may include instructions for keeping the LED of handpiece 106 in a light emitting state for a specific time frame (e.g., for 50 ms, 10 ms, 1 ms, etc.), then keeping the LED in a non-emitting state for a recovery period (e.g., a time frame ten times the duration of the time the LED was emitting light, a time frame fifty or one hundred times the duration of the time the LED was emitting light, a time frame longer than the duration of the time the LED was emitting light, etc.).

Controller 104 may be configured to control the therapeutic light provided to treatment region 108. For example, the therapeutic light may be provided at an average power density of about 100 W/cm² 200 W/cm² or more at treatment region 108. According to various exemplary embodiment, the therapeutic light may be provided at an average power density of about 500 W/cm², 1,000 W/cm², 5,000 W/cm², 10,000 W/cm², or 20,000 W/cm² or in the range of 200-20,000 is W/cm². Apparatus 100 including the LED may serve a an optical delivery system configured to direct the therapeutic light to treatment region 108 at an average power density specified by controller 104. In one embodiment, handpiece 106 includes a fiber optic configured to receive therapeutic light from the LED at a proximal end and to transmit the light to a distal end for illuminating treatment region 108.

Controller 104 may receive data from handpiece 106 via link 112 regarding LED usage. Data may include sensor data from a sensor within handpiece 106, data from the accelerometer of handpiece 106, or from another source. For example, controller 104 may receive data indicating that the LED has been emitting light for too long and is at risk of failure, and controller 104 may send instructions to the LED to turn off as a result. As another example, the accelerometer of handpiece 106 may provide data relating to the position or velocity of handpiece 106. Controller 104 may receive the data and may determine to change the state of the LED of handpiece 106 to a reduced power or non-emitting state if, for example, if the accelerometer indicates handpiece 105 has been stationary for a given period, of time emitting therapeutic light in a specific area (indicating a risk of overexposure of the subject to emitted light).

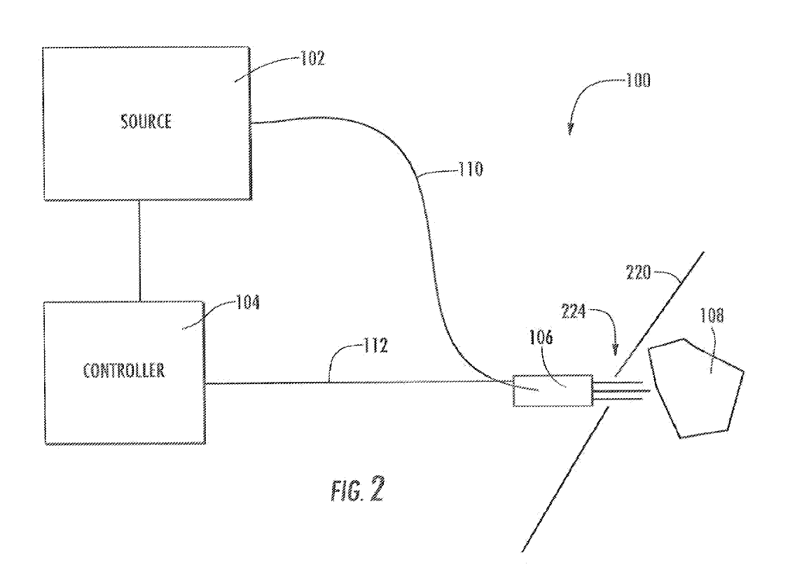

Referring to FIG. 2, the apparatus 100 for deliver mg therapeutic light to a treatment region is shown, according to another exemplary embodiment. In the embodiment of FIG. 2, apparatus 100 includes handpiece 106 providing therapeutic light to an internal treatment region 108. Handpiece 106 may include a surgical probe (e.g. a cannula) which may be inserted into a patient, e.g., through incision 224 in a skin surface 220. Optical fiber 112 may extent through the handpiece to a distal end of the probe to transmit therapeutic light to the treatment region.

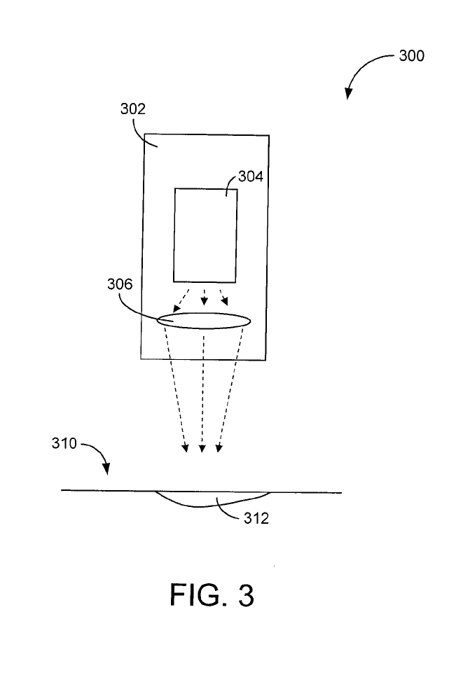

Referring now to FIG. 3, an apparatus 300 for providing therapeutic light to a treatment region is shown, according to yet another exemplary embodiment. Apparatus 300 includes flashlight or handpiece 302 shown in greater detail. Flashlight 302 includes an LED source 304 providing as light source through lens 306 to treatment region 310. LED source 304 may include one or more LEDs configured to provide a therapeutic light source to treatment region 310.

Lens 306 may be used to reflect the therapeutic light provided by LED source 304 for treatment region 310 such that the light directly (or indirectly, if desired) hits the surface of region 310. According to various exemplary embodiments, other optical elements (e.g. reflective elements, refractive elements, collimating elements, concentrating elements, diffusing elements, diffractive elements etc.) in addition to or in place of lens 306 may be used. The optical element may be used to direct therapeutic light to the treatment region with any desired patter, e.g. in a two dimensional pattern having regions of relatively high intensity and regions of relatively low intensity (e.g. as described in U.S. patent application Ser. No. 11.1347672 fled Feb. 3, 2006, the entire contents of which are incorporated by reference herein.

Treatment region 310 may include an area 312 below the surface of region 310. It may be desired for apparatus 300 to provide therapeutic light for area 312 below the surface of region 310, and the intensity and/or pattern of the light provided by apparatus 300 may be adjusted such that area 312 receives more therapeutic light. For example, lens 306 may be adjusted, the power provided to LED source 304 may be increased, or otherwise.

According to an exemplary embodiment, apparatus 300 or a portion of apparatus 300 may be configured to be inserted in treatment region 310 and area 312 such that an internal region (e.g., area 312) may receive the therapeutic light.

According to various exemplary embodiments, apparatus 300 may further include a controller or power source as described in FIGS. 1-2 such that a power supply for apparatus 300 or a configuration for how to provide therapeutic light may be provided within apparatus 300 instead of receiving power from an outside source or a configuration from an outside controller. As in the previously described embodiments, apparatus 300 may include one or more sensors (temperature sensors, inertial sensors, etc) which can provide information to controller, which, in turn can control the application of therapeutic light based on the information.

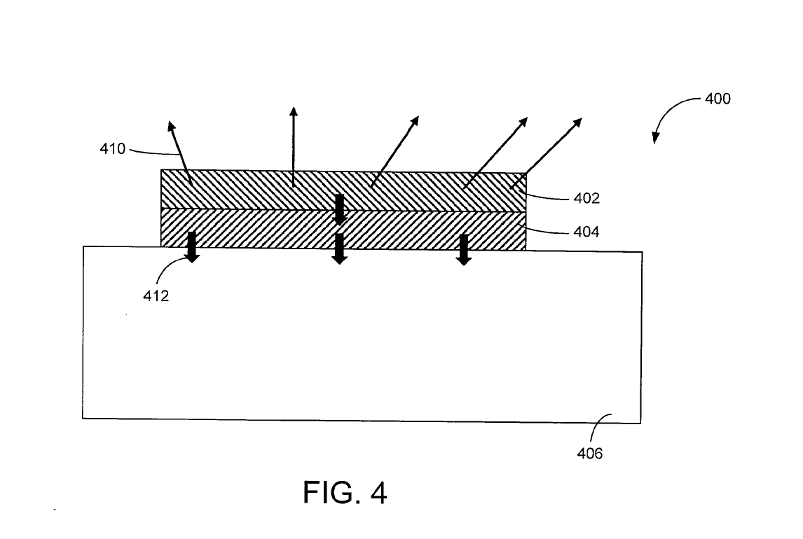

Referring to FIG. 4, a light source 400 for providing therapeutic light is shown in greater detail, according to an exemplary embodiment. Light source 400 includes an LED 402, a support substrate 404, and a heat sink 406. LED 402 may be an LED for providing therapeutic light as described in the present disclosure. LED 402 is shown emitting therapeutic light 410.

As indicated by the broad arrows, support substrate 404 may be used to accumulate the heat 412 generated by LED 402, and heat sink 406 may be used to dissipate, the heat in substrate 404. According to another exemplary embodiment, light source 400 may include multiple heat sinks instead of a single heat sink. According to various exemplary embodiments, other techniques may be used to provide a dissipation of heat generated by LED 402. For example, fluid circulated through heat sink 406 may be used to help dissipate the heat. In other embodiments, an active element such as Peltier cooler may be used, etc.

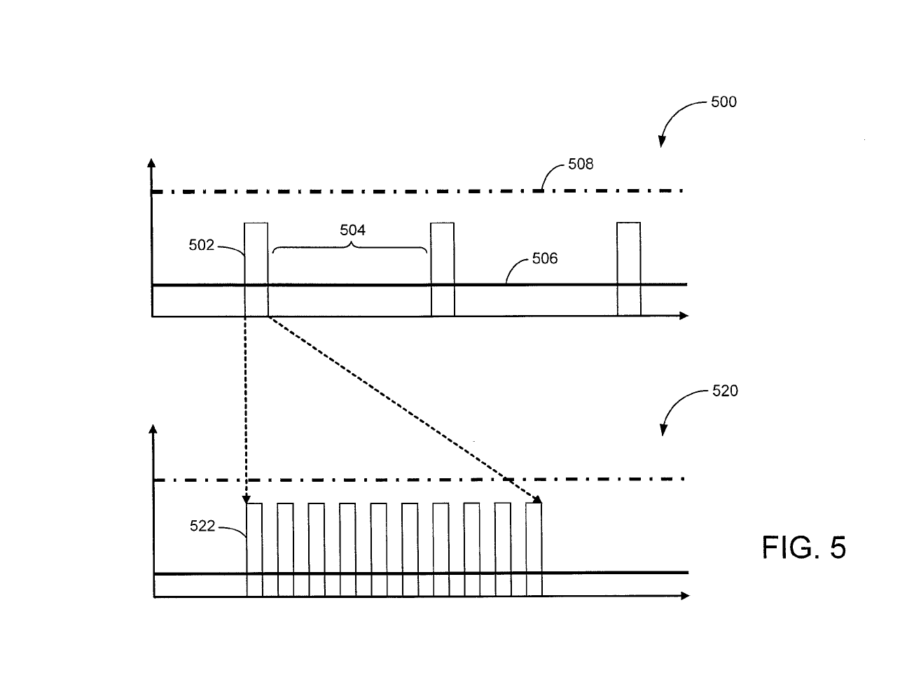

Retelling now to FIG. 5, graphs illustrating a pulse period for an LED is shown, according to an exemplary embodiment. Graph 500 illustrates a short pulse period 502 where the LED is in a light emitting state followed by a longer recovery period 504 under which the LED is in a non-emitting state. According to an exemplary embodiment, pulse period 502 may be 50 ms or less, 10 ms or less, 1 ms or less, or another time frame, and the duration of recovery period 504 may be ten times longer than the duration of pulse period 502, fifty times longer than the duration of pulse period 502, one hundred times longer than the duration of pulse period 502, or another time frame. The length of pulse period 502 and period 504 may be determined or adjusted based on how long the LED requires to recover between pulses to avoid deleterious effects.

Referring further to graph 500, a continuous operation power level 506 and a catastrophic failure power level 508 are illustrated. Continuous power level 506 represents the power level of the LED for operation in a conventional continuously emitting mode. As shown, during pulse period 502, the LED is pulsed to power levels above the continuous operation power level, to provide increased emission of light. During recovery period 504, the LED is shut off (or, alternatively, operated in a reduced power state), and allowed to recover from being “overdriven” during the pulse period. During the period, heat is dissipated from the LED, to avoid overheating which would lead to failure or degradation in performance of the LED.

Catastrophic failure power level 508 may indicate a maximum power level above which the LED will fail. According to various exemplary embodiments, the LED may be configured to operate at a power level that is equal to about 30% or more of the catastrophic failure power level, equal to about 50% or more of the catastrophic failure power level, equal to about 70% or more of the catastrophic failure power level, equal to about 90% or more of the catastrophic failure power level, or equal to another power level intermediate between levels 506 and 508, or otherwise.

Referring further to FIG. 5, pulse period 502 may further be broken down into sub-periods such that the LED may be operated in a “burst mode” where the LED is on for very short periods of time (e.g., 0.05 to 0.1 ms) alternating with periods (e.g. an equally long periods) off time for a total burst pulse duration of between, for example, 0.5 and 50 ms. For example, also referring to graph 520, during pulse period 502, the LED may be repetitively cycled between a light emitting state and a non-emitting state to produce a series of sub-pukes 522 of therapeutic light. The repetitive cycling may include, cycling the LED with a duty cycle of 50%, according to an exemplary embodiment. According to other exemplary embodiments, the duty cycle may be of any other value (e.g. 10%, 25%, 75% etc.) and the sub-pulses may be of varying length (e.g., each sub-pulse may be 1 ms or less). Burst mode operation may further reduce the stress on the LED while operating in at “overdriven” power levels greater than level 506.

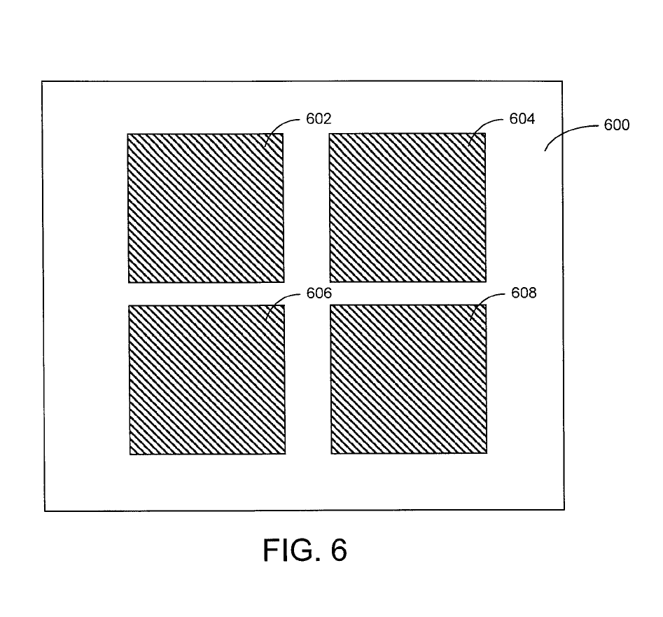

Referring now to FIG. 6, a block diagram of an array of light sources (e.g., multiple individual LEDs of the type described herein, etc) are shown, according to an exemplary embodiment. A device 600 may include multiple light sources 602, 604, 606, 608. According to various exemplary embodiments, device 600 may include any number of light sources and may include any number of rows or other configurations of light sources. For example, device 600 may be of any shape, may include less than four light sources, six light sources, eight light sources, or more, and may arrange the light sources in any configuration (e.g., all in a row, in a matrix form, circular form, or otherwise). Sources 602, 604, 606, 608 may be configured to be driven by a single controller for maintaining a pulse period and a non-emitting state for each light source, according to exemplary embodiments.

In the embodiment of FIG. 6, device 600 may be configured to provide therapeutic light from only one or some of the light sources 602, 604, 606, 608 at any given time. For example, device 600 may only provide therapeutic light from one light source at a time. Light may be provided by source 602 for a short period of time before entering a non-emitting state. Once source 602 shuts down for a cool down period, source 604 may begin providing therapeutic belt until source 604 must enter a non-emitting state. Source 606 and source 608 may then provide therapeutic light in a similar matter, and once source 608 shuts down for its non-emitting state, source 602 may be ready to provide therapeutic light again, therefore providing a cycle such that one light source may be providing therapeutic light at any given time.

According to various exemplary embodiments, only one light source at a time may provide therapeutic light in device 600. According to another exemplary embodiment, more than one light source may provide therapeutic light in device 600. According to yet another exemplary embodiment, one light source may provide therapeutic light some of the time, and none of the light sources may provide therapeutic light for another period of time. For example, in the embodiment of FIG. 6, if each sources 602, 604, 606, 608 require a down time that is ten times longer than the time frame in which each source provides light, device 600 may only provide therapeutic light for 40% of the time, while the other 60% of the time is spent with each of four sources 602, 604, 606, 608 in down time.

As will be understood by one skilled in the art, light sources of the type described herein may be used to provide therapeutic light for any suitable application. For example, dermatological treatments that may be administered using the systems and methods of the present disclosure may include, for example, facial rejuvenation, pigmented lesions, vascular lesions, acne and other dermatological applications for which a laser or IPL has been found to be effective. In one embodiment, the LED based device of the present disclosure could be used like a laser or IPL by a highly skilled practitioner, e.g. in a limited number of sessions a few weeks apart. In addition, a method of treatment with a LED based device with a lower peak power is suitable for a home use application with daily treatments for extended periods of time (e.g., weeks or months). For such method of treatment the accumulation of the effect from the multiple treatments may compensate for the lower peak power used in the individual treatment.

While this invention has been particular shown and described with references to example embodiments thereof, it will be understood by those skilled in the art that various changes in form and details may be made therein without departing from the scope of the invention encompassed by the appended claims.

One or more of any part thereof of the systems and methods of providing therapeutic light may be implemented in computer hardware or software, or a combination of both. The methods may be implemented in computer programs using standard programming techniques following the systems and methods described herein. Program code is applied to input data to perform the functions described herein and generate output information. The output information is applied to one or more output devices such as a display monitor. Each program may be implemented in a high level procedural or object oriented programming language to communicate with a computer system. However, the programs can be implemented in assembly or machine language, if desired. In any ease, the language can be a compiled or interpreted language. Moreover, the program can run on dedicated integrated circuits preprogrammed for that purpose.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during; program execution. Any analysis method can also be implemented as a computer-readable storage medium, configured with a computer program, where the storage medium so configured causes a computer to operate in a specific and predefined manner to perform the functions described herein.

What is claimed is:

1. A method of delivering therapeutic light to a treatment region to effect heating in the treatment region comprising:
a) providing a light emitting diode;
b) driving the light emitting diode to generate therapeutic light during a pulse period;
c) directing the therapeutic light to the treatment region at an average power density of about 200 W/cm̂2 or more at the treatment region during the pulse period;
d) maintaining the light emitting diode in a non-emitting state for a recovery period which is longer than the pulse period.

2. The method of claim 1, wherein the pulse period is about 50 ms or less.

3. The method any preceding claim, wherein the pulse period is about 10 ms or less.

4. The method of any preceding claim, wherein the pulse period is about 1 MS or less.

5. The method of any preceding claim, wherein the duration of the recovery period is at least 10 times the duration of the pulse period.

6. The method of any preceding claim, wherein the duration of the recovery period is at least 50 times the duration of the pulse period.

7. The method of any preceding claim, wherein the duration of the recovery period is at least 100 times the duration of the pulse period.

8. The method of any preceding claim further comprising:
during the pulse period, repetitively cycling the light emitting diode between a light emitting state and a non-emitting state to produce a series of sub-pulses of therapeutic light.

9. The method of claim 8, wherein the step of repetitively cycling comprises repetitively cycling the light emitting diode with a duty cycle of about 50% or less.

10. The method of claim 8 or claim 9, wherein the subpulses have a duration of about 1 millisecond or less.

11. The method of claim 8, 9, or 10, wherein the light emitting diode is characterized by a catastrophic failure power level, and wherein, during the subpulses, the light emitting diode is operated at a power level equal to about 30% or more of the catastrophic failure power level.

12. The method of claim 8, 9, or 10, wherein the light emitting diode is characterized by a catastrophic failure power level, and wherein, during the subpulses, the light emitting diode is operated at a power level equal to about 70% or more of the catastrophic failure power level.

13. The method of claim 8, 9, 10 or 11, wherein the light emitting diode is characterized by a catastrophic failure power level, and wherein, during the subpulses, the light emitting diode is operated at a power level equal to about 90% or more of the catastrophic failure power level.

14. The method of any preceding claim wherein the step of directing the therapeutic light to the treatment region at an average power density of about 200 W/cm̂2 or more at the treatment region during the pulse period comprises directing the therapeutic light to the treatment region at an average power density of about 500 W/cm̂2 or more at the treatment region during the pulse period.

15. The method of any preceding claim wherein the step of directing the therapeutic light to the treatment region at an average power density of about 200 W/cm̂2 or more at the treatment region during the pulse period comprises directing the therapeutic light to the treatment region at an average power density of about 1000 W/cm̂2 or more at the treatment region during the pulse period.

16. The method of any preceding claim wherein the step of directing the therapeutic light to the treatment region at an average power density of about 200 W/cm̂2 or more at the treatment region daring the pulse period comprises directing the therapeutic light to the treatment region at an average power density of about 5,000 W/cm̂2 or more tit the treatment region during the pulse period.

17. The method of any preceding claim wherein the step of directing the therapeutic light to the treatment region at an average power density of about 200 W/cm̂2 or more at the treatment region during the pulse period comprises directing the therapeutic light to the treatment region at an average power density of about 10,000 W/cm̂2 or more at the treatment region during the pulse period.

18. The method of any preceding claim wherein the step of directing the therapeutic light to the treatment region at an average power density of about 200 W/cm̂2 or more at the treatment region during the pulse period comprises directing the therapeutic light to the treatment region at an average power density of about 20,000 W/cm̂2 or more at the treatment region during the pulse period.

19. The method of any preceding claim further comprising dissipating heat generated by the light emitting diode using at least one heat sink in thermal communication with the light emitting diode.

20. The method of any preceding claim, wherein the therapeutic light comprises infrared light.

21. The method of any preceding claim further comprising effecting heating of the treatment region using the therapeutic light.

22. The method of claim 21, wherein the treatment region includes tissue, and wherein the heating results in a chemical or physical change to the tissue.

23. The method of claim 22, wherein the chemical or physical change comprises at least one selected from the list consisting of: ablation, cauterization, melting, shrinkage, lipolysis, and thermal damage.

24. The method of claim 22, wherein the tissue comprises collagen, and the chemical or physical change comprises heat induced collagen shrinkage.

25. The method of any preceding claim, wherein the treatment region is an external region of a human or animal subject.

26. The method of any preceding claim, wherein the treatment region is an internal region of a human or animal subject.

27. The method of any preceding claim, wherein the target region has a characteristic size of 1 cm or greater.

28. An apparatus for delivering therapeutic light, to a treatment region to effect heating in the treatment region comprising:
a light emitting diode;
a controller configured to drive the light emitting diode to generate therapeutic light during a pulse period and maintaining the light emitting diode in a non-emitting state for a recovery period which is longer than the pulse period;
an optical delivery system configured to direct the therapeutic light to the treatment region at an average power density of about 200 W/cm̂2 or more at the treatment region during the pulse period.

29. The apparatus of dam 28, wherein the pulse period is about 50 ms or less.

30. The apparatus of claim 28 or 29, wherein the pulse period is about 10 ms or less.

31. The apparatus of any of claims 28-30, wherein the pulse period is about 1 ms or less.

32. The apparatus of any of claims 28-31, wherein the duration of the recovery period is at least 10 times the duration of the pulse period.

33. The apparatus of any of claims 28-32, wherein the duration of the recovery period is at least 50 times the duration of the pulse period.

34. The apparatus of any of claims 28-33, wherein the duration of the recovery period is at least 100 times the duration of the pulse period.

35. The apparatus of any of claims 28-34:
wherein the controller is configured to drive the light emitting diode to, during the pulse period, repetitively cycle the light emitting diode between a light emitting state and a non-emitting state to produce a series of sub-pulses of therapeutic light.

36. The apparatus of claim 35, wherein the controller is configured to drive the light emitting diode to repetitively cycle the light emitting diode between the emitting and non-emitting states with a duty cycle of about 50% or less during the pulse period.

37. The apparatus of claim 35 or 36, wherein the subpulses have a duration of about 1 millisecond or less.

38. The apparatus of claim 35, 36 or 37, wherein the light emitting diode is characterized by a catastrophic failure power level, and wherein, during the subpulses, the light emitting diode is operated at a power level equal to about 30% or more of the catastrophic failure power level.

39. The apparatus of claim 35, 36, 37 or 38, wherein the light emitting diode is characterized by a catastrophic failure power level, and wherein, during the subpulses, the light emitting diode is operated at a power level equal to about 70% or more of the catastrophic failure power level.

40. The apparatus of claim 35, 36, 37, 38 or 39, wherein the light emitting diode is characterized by a catastrophic failure power level, and wherein, during the subpulses, the light emitting diode is operated at a power level equal to about 90% or more of the catastrophic failure power level.

41. The apparatus of any of claims 28-40, wherein the optical delivery system directs the therapeutic light to the treatment region at an average power density of about 500 W/cm̂2 or more at the treatment region during the pulse period.

42. The apparatus of any of claims 28-41, wherein the optical delivery system directs the therapeutic light to the treatment region at an average power density of about 1000 W/cm̂2 or more at the treatment region during the pulse period.

43. The apparatus of any of claims 28-42, wherein the optical delivery system directs the therapeutic light to the treatment region at an average power density of about 5,000 W/cm̂2 or more at the treatment region during the pulse period.

44. The apparatus of any of claims 28-43, wherein the optical delivery system directs the therapeutic light to the treatment region at an average power density of about 10,000 W/cm̂2 or more at the treatment region during the pulse period.

45. The apparatus of any of claims 28-44, wherein the optical delivery system directs the therapeutic light to the treatment region at an average power density of about 20,000 W/cm̂2 or more at the treatment region during the pulse period.

46. The apparatus of any of claims 28-45, further comprising at least one heat sink in thermal communication with the light emitting diode for dissipating heat generated by the light emitting diode.

47. The apparatus of any of claims 28-46, wherein the therapeutic light comprises infrared light.

48. The apparatus of any of claims 28-47, wherein the optical delivery system comprises a fiber optic configured to receive therapeutic light from the light emitting diode at a proximal end and transmit the light to a distal end to illuminate the treatment region.

49. The apparatus of any of claims 28-48, comprising a surgical handpiece which is configured for insertion into a human or animal subject to deliver therapeutic light to an internal region.

50. The apparatus of any of claims 28-49, comprising a handpiece for delivering therapeutic light to an external region of a human or animal subject.

51. The apparatus of any of claims 28-50, wherein the target region has a characteristic size of 1 cm or greater.

52. An apparatus thr delivering therapeutic light to a treatment region to effect heating in the treatment region comprising:
a plurality light of emitting diodes;
a controller configured to drive each respective one of the plurality of the light emitting diodes to generate therapeutic light during a respective pulse period and maintaining the respective light emitting diode in a non-emitting state for a respective recovery period which is longer than the pulse period,
at least one optical delivery system configured to direct the therapeutic light from the plurality of light emitting diodes to the treatment region at an average power density of about 200 W/cm̂2 at the treatment region during a treatment period.

53. The apparatus of claim 52, wherein the respective pulse periods of the respective light emitting diodes are staggered relative to each other during the treatment period.

54. The apparatus of claim 53, wherein at least one of the plurality of diodes in its respective pulse period daring substantially the entire treatment period.

55. The apparatus of any of claims 52-54, wherein the duration of the respective pulse period of each light emitting diode is 50 ms or less.

56. The apparatus of any of claims 52-54, wherein the duration of the respective pulse period of each light emitting diode is about 10 times the duration of the recovery period or more.
